(19) United States
(12) Patent Application Publication
(10) Pub. No.: US 2026/0165940 A1
(43) Pub. Date: Jun. 18, 2026

(54) OIL-BASED COSMETIC

(71) Applicant: SHISEIDO COMPANY, LTD., Chuo-ku, Tokyo (JP)

(72) Inventors: Hiroshi OKAMOTO, Tokyo (JP); Yusuke NAKANO, Tokyo (JP); Keisuke HAYASHIDA, Tokyo (JP)

(73) Assignee: SHISEIDO COMPANY, LTD., Chuo-ku, Tokyo (JP)

(21) Appl. No.: 18/713,428

(22) PCT Filed: Dec. 9, 2022
(86) PCT No.: PCT/JP2022/045389

§ 371 Date: May 24, 2024

(30) Foreign Application Priority Data
Dec. 24, 2021 (JP) 2021-210086

(51) Int. Cl. A61K 8/891; A61K 8/37; A61K 8/58; A61Q 19/00

(52) U.S. Cl. CPC A61K 8/891; A61K 8/37; A61K 8/585; A61Q 19/00; A61K 2800/10; A61K 2800/48

(57) ABSTRACT

The purpose of the present invention is to provide an oil-based cosmetic that exhibits long-term durability of an applied film, imparts neither a sticky feeling immediately after application nor a tight feeling after application, and imparts an excellent texture during application. The present invention relates to an oil-based cosmetic comprising a nonvolatile oil component and a volatile oil component. The oil-based cosmetic is characterized in that the nonvolatile oil component contains: (A) a silicone resin; (B) at least 10 mass % of trimethylpentaphenyltrisiloxane with respect to the total amount of the cosmetic; and (C) an ester oil having a viscosity of at least 100 mPa·s.

### TECHNICAL FIELD

The present invention relates to an oil-based cosmetic, more specifically to an oil-based cosmetic with excellent long-term durability of an applied film and excellent texture.

### BACKGROUND ART

Conventionally, ingredients with film-forming performance have been blended to strengthen applied films for the purpose of improving long-lasting properties of cosmetics. Among the ingredients with film-forming performance, silicone resin is often used because of its good compatibility with a volatile oil.

The silicone resin is a resin composed of siloxane bonds, and units constituting the backbone of siloxane include an M unit (monofunctional), a D unit (bifunctional), a T unit (trifunctional), and a Q unit (tetrafunctional) according to the number of functional groups. There are an MQ resin, an MT resin, an MD resin, and the like obtained by arbitrarily combining these units. In general, there is a tendency that a harder film is formed by using a resin containing a unit having a larger number of functional groups, while a flexible film is formed by using a resin containing a unit having a smaller number of functional groups. Therefore, when silicone resin is used as a film-forming agent, it is known that the use of an MQ resin containing Q units results in a robust film and an improvement in long-lasting properties of cosmetics. However, the addition of the MQ resin has sometimes caused discomfort such as stickiness and dryness after application to the skin.

For example, Patent Document 1 describes that a specific phenylated oil as a non-volatile oil is combined with a glyceryl ester, silica, or wax as a gelling agent by using polypropylsilsesquioxane, a silicone resin containing T units, to provide a cosmetic with improved long-term durability and transfer resistance.

On the other hand, a cosmetic using silicone resin as a film-forming agent sometimes gives a feeling of tightness or dryness after application to the skin when the quantity of a volatile oil together with the silicone resin increases. Depending on the type of silicone resin to be used and the oil to be combined, the film-forming property may deteriorate, or the texture after application may become poor. Thus, it has been difficult to achieve both excellent film-forming property and good texture.

### CITATION LIST

### Patent Document

Patent Document 1: JP-B 6038952

### SUMMARY OF INVENTION

### Technical Problem

An object of the present invention is to provide an oil-based cosmetic that exhibits excellent texture with little stickiness immediately after application and little dryness after application, while having long-term durability of an applied film.

### Solution to Problem

As a result of intensive studies to solve the problem above, the present inventors have found that a silicone resin is blended with a specific non-volatile oil to provide an oil-based cosmetic that has long-term durability of an applied film and gives excellent texture with no stickiness immediately after application and no dryness after application, thus completing the present invention.

Specifically, the present invention provides an oil-based cosmetic comprising a non-volatile oil and a volatile oil, wherein the non-volatile oil comprises:
(A) a silicone resin,
(B) 10% by mass or more with respect to the total amount of the cosmetic of trimethylpentaphenyltrisiloxane, and
(C) an ester oil having a viscosity of 100 mPa·s or more.

### Advantageous Effects of Invention

The oil-based cosmetic of the present invention has improved film-forming properties and enhanced long-term durability of the applied film by blending (A) a silicone resin with a non-volatile oil containing a combination of (B) trimethylpentaphenyltrisiloxane and (C) an ester oil. As a result, long-lasting property of cosmetic is improved. Furthermore, the cosmetic can give an excellent texture without a feeling of stickiness immediately after application or dryness (feeling of tightness due to drying) after application.

### DESCRIPTION OF EMBODIMENTS

The oil-based cosmetic of the present invention contains a non-volatile oil and a volatile oil. The “non-volatile oil” is, for example, an oily component remaining on the skin at room temperature and atmospheric pressure (normal temperature and normal pressure). In the present specification, an oily component having an evaporation rate of less than 0.01 mg/cm²/min at normal temperature and normal pressure is referred to as a “non-volatile oil”.

The oil-based cosmetic of the present invention contains the non-volatile oil containing a combination of (A) a silicone resin, a predetermined amount of (B) trimethyl pentaphenyl trisiloxane, and (C) an ester oil having a specific viscosity, which will be described in detail below.

### (A) Silicone Resin

The silicone resin (A) used in the oil-based cosmetic of the present invention (hereinafter sometimes referred to as “component A”) is not particularly limited as long as it can commonly be used in cosmetics. In other words, the silicone resin (A) may be composed of any combination of trialkylsiloxy units (M units (R₃SiO_{1/2})), dialkylsiloxy units (D units (R₂SiO_{2/2})), monoalkylsiloxy units (T units (RSiO_{3/2})), and tetrafunctional siloxy units (Q units (SiO_{4/2})). Specific examples thereof include solid silicone reticulate compounds called MQ resin, MT resin, MDQ resin, MTQ resin, MDTQ resin, TD resin, TQ resin, and TDQ resin. Substituents (R) bonded to silicon of these silicone resins each independently represent a hydrocarbon substituent such as an alkyl group, a phenyl group, an alkenyl group, and an aryl group, and the hydrocarbon substituent preferably has 1 to 10 carbon atoms. Some or all of the hydrogen atoms may be substituted with fluorine atoms or other organic functional groups.

Examples of the MQ resin (silicone resin composed of M and Q units) include trimethylsiloxysilicate, trifluoroalkyldimethyl/trimethylsiloxysilicate, and phenylpropyldimethylsiloxysilicate, which are commercially available, such as X-21-5250 (trimethylsiloxysilicate, 50% cyclopentasiloxane solution), KF-7312T (trimethylsiloxysilicate, 60% methyl trimethicone solution), KF-7312J (trimethylsiloxysilicate, 50% cyclopentasiloxane solution), KF-7312K (trimethylsiloxysilicate, 60% dimethicone solution), and KF-9021 (trimethylsiloxysilicate, 50% cyclopentasiloxane solution) (all manufactured by Shin Etsu Chemical Co Ltd.); SR 1000 (trimethylsiloxysilicate, manufactured by Momentive Performance Materials, Inc.); XS66-B8636 (trifluoroalkyldimethyl/trimethylsiloxysilicate, 50% dimethicone solution, manufactured by Momentive Performance Materials, Inc.); and Silshine 151 (phenylpropyldimethylsiloxysilicate, manufactured by Momentive Performance Materials, Inc.). The MQ resin to be used as component A in the oil-based cosmetic of the present invention is preferably trimethylsiloxysilicate.

Examples of the silicone resin containing at least one T unit (also referred to herein as “MT resin”) include polymethylsilsesquioxane, polypropylsilsesquioxane, polyphenylsilsesquioxane, and trifluoropropyldimethylsiloxy/trimethylsiloxy silsesquioxane, which are commercially available, such as SILFORM FLEXIBLE RESIN (Polymethylsilsesquioxane, manufactured by Momentive Performance Materials, Inc.), DOWSIL 670 Fluid (polypropylsilsesquioxane, manufactured by Dow Corning Toray Co., Ltd.), BELSIL PMK POWDER (polymethylsilsesquioxane, manufactured by Wacker Asahikasei Silicone Co., Ltd.), KR-220L (polymethylsilsesquioxane, manufactured by Shin Etsu Chemical Co Ltd.), BELSIL SPR 45 VP (polyphenylsilsesquioxane, manufactured by Wacker Asahikasei Silicone Co., Ltd.), and FR-5 (trifluoropropyldimethylsiloxy/trimethylsiloxy silsesquioxane, manufactured by Momentive Performance Materials, Inc.).

Component A in the oil-based cosmetic of the present invention preferably contains a silicone resin selected from MQ and MT resins. Either the MQ resin or the MT resin may be blended alone, but the MQ resin is preferably blended. In the oil-based cosmetic of the present invention, it is preferable to use an MQ resin (particularly, trimethylsiloxysilicate) and an MT resin (particularly, polymethylsilsesquioxane) in combination.

The amount of the silicone resin (component A) in the oil-based cosmetic of the present invention is preferably 10 to 30% by mass, more preferably 15 to 25% by mass, based on the total amount of the cosmetic. The amount of the silicone resin (component A) is preferably in the range of 20 to 60% by mass, more preferably in the range of 30 to 50% by mass, based on the total amount of the non-volatile component contained in the cosmetic. When the amount of component A is less than 10% by mass of the total amount of the cosmetic or less than 20% by mass of the total amount of the non-volatile component, the film-forming property may become poor. When the amount of component A is more than 30% by mass of the total amount of the cosmetic or more than 60% by mass of the total amount of the non-volatile component, a comfortable texture tends to be lost.

### (B) Trimethylpentaphenyltrisiloxane

The trimethylpentaphenyltrisiloxane (B) (hereinafter sometimes referred to as “component B”) in the oil-based cosmetic of the present invention is one phenyl-modified silicone usually used in cosmetics, which is also commercially available, such as Dow PH-1555 HRI Cosmetic Fluid (manufactured by Dow Corning Toray Co., Ltd.).

The amount of the trimethylpentaphenyltrisiloxane (component B) in the oil-based cosmetic of the present invention is 10% by mass or more, preferably 21% by mass or more, and more preferably 30% by mass or more, based on the total amount of the cosmetic. The upper limit of the amount based on the total amount of the cosmetic is not particularly limited, but can be, for example, 60% by mass or less, preferably 50% by mass or less. The amount of the trimethylpentaphenyltrisiloxane (component B) may fall within the range of the lower limit and upper limit described above, for example, 10 to 60% by mass, 10 to 50% by mass, 21 to 60% by mass, 21 to 50% by mass, 30 to 60% by mass, and 30 to 50% by mass.

The amount of the trimethylpentaphenyltrisiloxane (component B) is preferably in the range of 20 to 60% by mass, more preferably in the range of 20 to 50% by mass, even more preferably 36 to 50% by mass, based on the total amount of the non-volatile component. When the amount of component B is less than 10% by mass of the total amount of the cosmetic or less than 20% by mass of the total amount of the non-volatile component, the applied film may be less likely to remain. When the amount of component B is more than 60% by mass of the total amount of the cosmetic or more than 60% by mass of the total amount of the non-volatile component, stickiness may occur.

### (C) Ester Oil

The ester oil (C) in the oil-based cosmetic of the present invention (hereinafter sometimes referred to as “component C”) is a non-volatile oil recognized as corresponding to an “ester oil” in the technical field of cosmetics, and is an oil having a viscosity of 100 mPa·s or more, preferably 420 mPa·s or more, and more preferably 1000 mPa·s or more at 20° C. (therefore, “component C” of the present invention is sometimes referred to as an “ester oil having a specific viscosity”). As used herein, the term “viscosity” refers to a value measured using a B-type viscometer.

Specific examples of the ester oil (component C) having a specific viscosity in the present invention include, but are not limited to, triisostearin (viscosity: 13890 mPa·s), trimethylolpropane triisostearate (viscosity: 257 mPa·s), pentaerythrityl tetraethylhexanoate (viscosity: 124 mPa·s), pentaerythrityl tetraisostearate (viscosity: 420 mPa·s), polyglyceryl-6 octacaprylate (viscosity: 369 mPa·s), diisostearyl malate (viscosity: 5500 mPa·s), polyglyceryl-2 triisostearate (viscosity: 448 mPa·s), and (isostearic acid/sebacic acid) ditrimethylolpropane oligoester (viscosity: 2380 mPa·s). In the present invention, diisostearyl malate is particularly preferably used.

The amount of the ester oil (component C) having a specific viscosity in the oil-based cosmetic of the present invention is 5 to 30% by mass, preferably 10 to 20% by mass, based on the total amount of the cosmetic. The amount is preferably in the range of 10 to 40% by mass, more preferably in the range of 10 to 20% by mass, based on the total amount of the non-volatile component. When the amount is less than 5% by mass based on the total amount of the cosmetic, a granular mass is likely to be generated, and when the amount is more than 30% by mass, the long-lasting property of the cosmetic may deteriorate.

The “non-volatile oil” in the oil-based cosmetic of the present invention may contain another oily component in addition to the essential components A, B and C.

For example, an oily (oil-soluble) thickener can be blended to adjust the viscosity of the cosmetic. As the oily thickener to be blended in the oil-based cosmetic of the present invention, a glyceryl ester is preferably used.

On the other hand, when a hydrocarbon wax such as polyethylene wax is blended in the cosmetic of the present invention, the object of the present invention such as long-lasting properties of cosmetics and texture can be achieved. However, a granular mass may be generated through interaction with other blended components. The term “granular mass” means a mass of the cosmetic in a granular form due to poor adhesion to the skin and poor spreadability when the cosmetic is applied, or a small mass of the cosmetic generated by cracking and partial peeling of a dry applied film over time.

The glyceryl ester, an oily thickener to be used in the oil-based cosmetic of the present invention, is not particularly limited as long as it is commonly used in cosmetics. Examples thereof include glyceryl tribehenate, glyceryl tristearate, glyceryl tripalmitate, glyceryl trilaurate, glyceryl behenate, glyceryl behenate/polyglyceryl-6 octastearate, and glyceryl behenate/eicosadioate, which are also commercially available, such as Estelide G-BP (glyceryl tribehenate, manufactured by National Mimatsu Co., Ltd.), Estelide G-S (glyceryl tristearate, manufactured by National Mimatsu Co., Ltd.), Estelide G-P (glyceryl tripalmitate, manufactured by National Mimatsu Co., Ltd.), Estelide G-L (glyceryl trilaurate, manufactured by National Mimatsu Co., Ltd.), Compritol 888 CG ATO (glyceryl tribehenate, manufactured by Gattefosse), TAISET 50-C (glyceryl behenate/polyglyceryl-6 octastearate, manufactured by Taiyo Kagaku Co., Ltd.), and NOMCORT HK-G (glyceryl behenate/eicosadioate, manufactured by The Nisshin OilliO Group, Ltd.). In the oil-based cosmetic of the present invention, glyceryl behenate/eicosadioate is preferred.

The amount of the glyceryl ester, an oily thickener to be used in the oil-based cosmetic of the present invention, is not particularly limited as long as it is an amount usually blended in cosmetics for the purpose of thickening. For example, the amount is preferably in the range of 1 to 10% by mass, more preferably in the range of 2 to 6% by mass, based on the total amount of the cosmetic.

Furthermore, other oily components satisfying the above conditions (the evaporation rate at normal temperature and normal pressure is less than 0.01 mg/cm²/min) can be blended as “non-volatile oils” within a range that does not impair the effects of the present invention. Examples thereof include hydrocarbon oils, higher fatty acids, higher alcohols, fats and oils, ester oils (other than component C), silicone oils (other than component B), oily pigments, and surfactants.

The oil-based cosmetic of the present invention may further contain a “powder”.

In the present invention, the “non-volatile oils” and the powder are collectively referred to as “non-volatile components”.

The powder to be used in the oil-based cosmetic of the present invention is not particularly limited as long as it is commonly used in cosmetics. Examples of such a powder include a spherical powder, a plate-like powder, and a colorant.

Examples of the spherical powder include porous spherical powder such as silica; and spherical resin powder such as polymethyl methacrylate, organopolysiloxane elastomer, polystyrene, polyamide resin (nylon), polyethylene, copolymer of styrene and acrylic acid, benzoguanamine resin, polytetrafluoroethylene, and silicone resin.

Examples of the plate-like powder include inorganic powders such as mica, synthetic mica (synthetic fluorphlogopite), talc, sericite, aluminum oxide, magnesium oxide, zirconium oxide, magnesium carbonate, calcium carbonate, calcium sulfate, chromium oxide, chromium hydroxide, aluminum silicate, magnesium silicate, aluminum magnesium silicate, kaolin, silicon carbide, barium sulfate, bentonite, smectite, and boron nitride; organic powders such as N-acyllysine; and composite powders such as particulate titanium oxide-coated mica titanium, particulate zinc oxide-coated mica titanium, and barium sulfate-coated mica titanium.

The colorant is an ingredient blended for the purpose of imparting the color of the cosmetic itself or the color when applied to the skin or lips, and specific examples thereof include organic pigments, color pigments, white pigments, pearl pigments, and glitter agents, which are generally blended in oil-based cosmetics.

Examples of the organic pigments include, but are not limited to, Lithol Rubine BCA (Red No. 202), Lake Red C (Red No. 203), Lake Red CBA (Red No. 204), Lithol Red (Red No. 205), Lithol Red CA (Red No. 206), Lithol Red BA (Red No. 207), Lithol Red SR (Red No. 208), Brilliant Lake Red R (Red No. 219), Deep Maroon (Red No. 220), Toluidine Red (Red No. 221), Permaton Red (Red No. 228), Permanent Orange (Orange No. 203), Benzidine Orange G (Orange No. 204), Benzidine Yellow G (Yellow No. 205), Brilliant Fast Scarlet (Red No. 404), Permanent Red F5R (Red No. 405), Hanza Orange (Orange No. 401), Hanza Yellow (Yellow No. 401), and Phthalocyanine Blue (Blue No. 404).

Examples of the color pigments include red iron oxide, iron titanate, Y-iron oxide, yellow iron oxide, loess, black iron oxide, carbon, low-order titanium oxide, mango violet, cobalt violet, chromium oxide, chromium hydroxide, cobalt titanate, ultramarine blue, and Prussian blue.

Examples of the white pigments include titanium dioxide, zinc oxide, and barium sulfate.

Examples of pearlescent pigments (pearl pigments) include titanium dioxide-coated mica (mica titanium), iron oxide-coated mica titanium, carmine coated mica titanium, carmine/blue coated mica titanium, iron oxide/carmine treated mica titanium, blue treated mica titanium, iron oxide/blue treated mica titanium, chromium oxide-treated mica titanium, black titanium oxide mica titanium, acrylic resin-coated aluminum powder, titanium oxide-coated mica, titanium oxide-coated bismuth oxychloride, titanium oxide-coated talc, colored titanium oxide-coated mica, titanium oxide-coated synthetic mica, titanium oxide-coated silica, titanium oxide-coated alumina, titanium oxide-coated glass powder, polyethylene terephthalate/polymethyl methacrylate laminated film powder, bismuth oxychloride, and fish scale foil.

As the glitter agent, resin or metal powder can be used, and examples thereof include polyethylene terephthalate/aluminum/epoxy laminate powder, polyethylene terephthalate/aluminum laminate powder, polyethylene terephthalate/gold laminate powder, polyethylene terephthalate/polyolefin laminate film powder, polyethylene terephthalate/polymethyl methacrylate laminate film powder, polyethylene/polyester laminate powder, polyethylene/polyethylene terephthalate laminate powder, and acrylic resin-coated aluminum powder. Furthermore, these powders colored with a legal dye or an inorganic pigment can also be used.

The amount of the powder to be used in the oil-based cosmetic of the present invention can be appropriately set according to the purpose of the oil-based cosmetic. From the viewpoint of film-forming property (strength of the cosmetic film), the amount of the powder excluding the colorant is preferably 8% by mass or less based on the total amount of the cosmetic.

The oil-based cosmetic of the present invention contains a “non-volatile component” obtained by adding an arbitrary powder to a non-volatile oil, and further contains a volatile oil. By blending the volatile oil, the spreadability of the cosmetic at the time of application becomes better.

The volatile oil blended in the cosmetic of the present invention may be any volatile oil commonly used in cosmetics and can be defined as, for example, an oil having a boiling point in the range of 60 to 260° C. at normal pressure. In the cosmetic of the present invention, it is preferable to select a volatile oil in which any of the essential components A, B and C are soluble. Examples thereof include a volatile hydrocarbon oil having 8 to 16 carbon atoms, a volatile silicone oil, and a mixture thereof. In particular, such volatile oils are selected from branched C8 to C16 alkanes, branched C8 to C16 esters, and mixtures thereof. Especially, isododecane or isohexadecane, isohexyl neopentanoate, and mixtures thereof are preferred, and even more preferred is isododecane. In order to achieve the maximum color maintaining property and good texture, the amount of the volatile oil is preferably 0.1 to 50% by mass, and more preferably 1 to 30% by mass, based on the total amount of the cosmetic.

In the oil-based cosmetic of the present invention, the thickening property and colorant dispersibility can be further improved according to the amount of the volatile oil. Specifically, when the amount of the volatile oil is 50% by mass or more based on the total amount of the cosmetic, it is preferable that the content of component A be 38% by mass or less, the content of component B is 25% by mass or less, and the content of component C is 25% by mass or more based on the non-volatile component.

The oil-based cosmetic of the present invention can be produced according to a conventionally used method, and the dosage form can be, for example, liquid, paste, solid, semi-solid, or gel. Above all, the oil-based cosmetic of the present invention is preferably in a liquid form or a paste form.

The oil-based cosmetic of the present invention can be provided in the form of, for example, lipsticks, lip glosses, lip bases, overcoats for lipsticks (lip coats), lip balms, eye glosses, blushes, foundations, and eyeshadows. In particular, when it used as a lipstick or eyeshadow, the oil-based cosmetic is likely to exhibit the advantageous effects of the present invention.

### Examples

The present invention is described in more detail below with reference to Examples, but the present invention is not limited to these Examples at all. Unless otherwise specified, the amount means % by mass based on the total amount of the cosmetic. Prior to Examples, evaluation methods used in the present invention are described.

### Evaluation Method

### 1. Remaining Degree of Applied Film (Color Maintaining Property)

A practical use test was conducted by a panel of 10 experts. Specifically, samples were each applied to the lips to score the long-lasting property of the cosmetic two hours after application based on the following scoring criteria. Moreover, the evaluation was made based on the following evaluation criteria from the total score.

### <Scoring Criteria>

5 points: Very good
4 points: Good
3 points: Fair
2 points: Poor
1 point: Very poor

### <Evaluation Criteria>

A: Total score of 40 or more
B: Total score of 35 to 39
C: Total score of 25 to 34
D: Total score of 24 or less

### 2. Texture (Feeling on Use)

A practical use test was conducted by a panel of 10 experts. The evaluation items were “no feeling of tightness” and “no feeling of stickiness (sticky feeling)”, and each evaluation item was scored based on the following scoring criteria. Moreover, the evaluation was made based on the following evaluation criteria from the total score.

### <Scoring Criteria>

5: Very good
4: Good
3: Fair
2: Poor
1: Very poor

### <Evaluation Criteria>

A: Total score of 40 or more
B: Total score of 35 to 39
C: Total score of 25 to 34
D: Total score of 24 or less

### 3. Generation of Granular Mass

A practical use test was conducted by a panel of 10 experts, and the appearance of each sample was visually observed and evaluated based on the following evaluation criteria.

### <Evaluation Criteria>

Generation of granular mass not observed=“No”
Generation of granular mass observed=“Yes”

Liquid lipsticks with the compositions listed in Tables 1 to 3 below were prepared according to conventional methods. According to the evaluation methods described above, the remaining degree of the applied film (color maintaining property), the texture (no feeling of tightness and no feeling of stickiness (sticky feeling)), and the generation of granular mass were evaluated. The results are also shown in each table.

TABLE 1

| | Exam- | Exam- | Exam- | Exam- |
|---|---|---|---|---|
| Ingredients | ple 1 | ple 2 | ple 3 | ple 4 |
| Glyceryl behenate/eicosadioate | 3 | 3 | 3 | 3 |
| Isododecane | 30 | 30 | 25 | 25 |
| Trimethylsiloxysilicate | 12 | 20 | 12 | 12 |
| Polymethylsilsesquioxane | 8 | — | 8 | 8 |
| Trimethyl pentaphenyl | 32.5 | 32.5 | 32.5 | 32.5 |
| trisiloxane | | | | |
| Diisostearyl malate | 10 | 10 | 10 | 10 |
| Red 202 | 2.16 | 2.16 | 2.16 | 2.16 |
| Barium sulfate | 2.34 | 2.34 | 2.34 | 2.34 |
| Silica | — | — | 5 | — |
| Synthetic fluorphlogopite | — | — | — | 5 |
| Total | 100 | 100 | 100 | 100 |
| Volatile component | 30 | 30 | 25 | 25 |
| Non-volatile component | 70 | 70 | 75 | 75 |
| Percentage of A in non-volatile | 28.6 | 28.6 | 26.7 | 26.7 |
| component (%) | | | | |
| Percentage of B in non-volatile | 46.4 | 46.4 | 43.3 | 43.3 |
| component (%) | | | | |
| Percentage of C in non-volatile | 14.3 | 14.3 | 13.3 | 13.3 |
| component (%) | | | | |
| Remaining of applied film (color | A | A | A | A |
| maintaining property) | | | | |
| No feeling of tightness | A | A | A | A |
| No feeling of stickiness | A | B | A | A |
| Generation of granular mass | No | No | No | No |

TABLE 2

| | Comparative | Example | Comparative | Example | Example |
|---|---|---|---|---|---|
| Ingredients | Example 1 | 5 | Example 2 | 6 | 7 |
| Glyceryl behenate/eicosadioate | 3 | 3 | 3 | 3 | 3 |
| Isododecane | 30 | 30 | 55 | 52 | 42 |
| Trimethylsiloxysilicate | 12 | 20 | 12 | 12 | 12 |
| Polymethylsilsesquioxane | 8 | — | 8 | 8 | 8 |
| Trimethyl pentaphenyl | 32.5 | 32.5 | 7.5 | 10.5 | 20.5 |
| trisiloxane | | | | | |
| Diisostearyl malate (5500 | — | — | 10 | 10 | 10 |
| mPa · s) | | | | | |
| Isopropyl myristate (7 mPa · s) | 10 | — | — | — | — |
| Pentaerythrityl Tetraisostearate | — | 10 | — | — | — |
| (420 mPa · s) | | | | | |
| Red 202 | 2.16 | 2.16 | 2.16 | 2.16 | 2.16 |
| Barium sulfate | 2.34 | 2.34 | 2.34 | 2.34 | 2.34 |
| Total | 100 | 100 | 100 | 100 | 100 |
| Volatile component | 30 | 30 | 55 | 52 | 42 |
| Non-volatile component | 70 | 70 | 45 | 48 | 58 |
| Percentage of A in non-volatile | 28.6 | 28.6 | 44.4 | 41.7 | 34.5 |
| component (%) | | | | | |
| Percentage of B in non-volatile | 46.4 | 46.4 | 16.7 | 21.9 | 35.3 |
| component (%) | | | | | |
| Percentage of C in non-volatile | 14.3 | 14.3 | 22.2 | 20.8 | 17.2 |
| component (%) | | | | | |
| Remaining of applied film | C | A | C | B | B |
| (color maintaining property) | | | | | |
| No feeling of tightness | A | A | A | A | A |
| No feeling of stickiness | A | A | B | A | A |
| Generation of granular mass | No | No | No | No | No |

TABLE 3

| | Example | Example |
|---|---|---|
| Ingredients | 8 | 9 |
| Glyceryl behenate/eicosadioate | 3 | — |
| Polyethylene wax | — | 3 |
| Isododecane | 27.6 | 27.6 |
| Trimethylsiloxysilicate | 12 | 12 |
| Polymethylsilsesquioxane | 8 | 8 |
| Trimethyl pentaphenyl trisiloxane | 32.5 | 32.5 |
| Diisostearyl malate | 10 | 10 |
| Polysorbate 60 | 2.4 | 2.4 |
| Red 202 | 2.16 | 2.16 |
| Barium sulfate | 2.34 | 2.34 |
| Total | 100 | 100 |
| Volatile component | 27.6 | 27.6 |
| Non-volatile component | 72.4 | 72.4 |
| Percentage of A in non-volatile component | 27.6 | 27.6 |
| (%) | | |
| Percentage of B in non-volatile component | 44.9 | 44.9 |
| (%) | | |
| Percentage of C in non-volatile component | 13.8 | 13.8 |
| (%) | | |
| Remaining of applied film (color maintaining | A | B |
| property) | | |
| No feeling of tightness | A | A |
| No feeling of stickiness | A | A |
| Generation of granular mass | No | Yes |

As shown in Tables 1 to 3, Examples 1 to 9 in which a non-volatile oil containing (A) a silicone resin, (B) trimethyl pentaphenyl trisiloxane, and (C) an ester oil having a specific viscosity was blended all had excellent ratings in terms of the remaining degree of the applied film (color maintaining property) and the texture (no feeling of tightness and non-sticky feeling). On the other hand, Comparative Example 1, which contained only an ester oil having a viscosity of less than 100 mPa·s, and Comparative Example 2, in which the amount of trimethyl pentaphenyl siloxane did not meet the predetermined requirement particularly exhibited poor remaining of the applied film (color maintaining property), resulting in poor long-lasting property of cosmetic.

Compared to Example 2, which contained only MQ resin as component A, Example 1, which contained MQ and MT resins in combination, had a tendency to provide an improved non-sticky feeling. In addition, Example 8, in which glyceryl ester was blended as an oily thickener, generated no granular mass, while Example 9, in which polyethylene wax was blended, generated granular mass.

Formulation Examples of the cosmetic of the present invention are given below. The present invention is not limited by Formulation Examples at all and is of course specified by the claims.

Formulation Example 1: Liquid Lipstick

| (Ingredient) | Amount (% by mass) |
|---|---|
| Glyceryl behenate/eicosadioate | 3 |
| Isododecane | Balance |
| Polymethylsilsesquioxane | 8 |
| Trimethylsiloxysilicate | 8 |
| Trimethyl pentaphenyl trisiloxane | 32.5 |
| Diisostearyl malate | 10 |
| Red 202 | 2.16 |
| Barium sulfate | 2.34 |
| Nicotinic acid amide | 5 |
| Total | 100 |

Formulation Example 2: Liquid Lipstick

| (Ingredient) | Amount (% by mass) |
|---|---|
| Glyceryl behenate/eicosadioate | 3 |
| Isododecane | Balance |
| Polymethylsilsesquioxane | 8 |
| Trimethylsiloxysilicate | 8 |
| Trimethyl pentaphenyl trisiloxane | 32.5 |
| Diisostearyl malate | 10 |
| Red 202 | 2.16 |
| Barium sulfate | 2.34 |
| Nicotinic acid amide | 0.1 |
| Total | 100 |

1. An oil-based cosmetic comprising a non-volatile oil and a volatile oil,
wherein the non-volatile oil comprises:
(A) 10 to 30% by mass with respect to the total amount of the cosmetic of a silicone resin,
(B) 10 to 60% by mass or more with respect to the total amount of the cosmetic of trimethylpentaphenyltrisiloxane, and
(C) an ester oil having a viscosity of 100 mPa·s or more.

2. The oil-based cosmetic according to claim 1, wherein the silicone resin (A) is a combination of an MQ resin and a silicone resin containing at least one T unit.

3. The oil-based cosmetic according to claim 2, wherein the MQ resin is trimethylsiloxysilicate, and the silicone resin containing at least one T unit is polymethylsilsesquioxane.

4. The oil-based cosmetic according to claim 1, wherein the ester oil (C) is an ester oil having a viscosity of 1000 mPa·s or more.

5. The oil-based cosmetic according to claim 4, wherein the ester oil (C) is diisostearyl malate.

6. The oil-based cosmetic according to claim 1, further comprising a glyceryl ester as an oily thickener.

7. The oil-based cosmetic according to claim 1, further comprising a powder, wherein an amount of the powder excluding a colorant is 8% by mass or less.
